(19) United States
(12) Patent Application Publication
(10) Pub. No.: US 2014/0358130 A1
(43) Pub. Date: Dec. 4, 2014

(54) INSTRUMENT USE COUNTER

(71) Applicant: Zimmer, Inc., Warsaw, IN (US)

(72) Inventor: Timothy James Gardner, Fort Wayne, IN (US)

(73) Assignee: Zimmer, Inc., Warsaw, IN (US)

(21) Appl. No.: 14/285,015

(22) Filed: May 22, 2014

Related U.S. Application Data
(60) Provisional application No. 61/827,892, filed on May 28, 2013.

(51) Int. Cl. A61B 19/02; A61B 19/00

(52) U.S. Cl. CPC A61B 19/0256; A61B 19/00; A61B 2560/028; A61B 2019/4815
USPC 606/1; 206/570; 206/370

(57) ABSTRACT

An instrument use counter and related method are disclosed. The instrument use counter can include a tray, an instrument holder, an actuator, and a counter. The tray can include an instrument holder positioned on or above an upper surface of the tray. The instrument holder can be configured to retain at least one instrument. The actuator can be located in proximity to the instrument holder and can include a proximity sensor in electronic communication with the at least one instrument or a moveable member engageable with the at least one instrument during removal from, or receipt by, the instrument holder. The counter can be coupled to the tray, positioned above the upper surface, and operably coupled to the proximity sensor or the movable member of the actuator. The counter can include a numerical readout member that can track instrument use.

### CLAIM OF PRIORITY

This patent application claims the benefit of priority to U.S. Provisional Patent Application Ser. No. 61/827,892, titled “INSTRUMENT USE COUNTER” to Gardner and filed on May 28, 2013, which is hereby incorporated by reference herein in its entirety.

### TECHNICAL FIELD

The present disclosure relates to surgical accessories.

### BACKGROUND

In instrument or tool use applications, instrument readiness can be extremely important and some instruments must be maintained in excellent working order and condition. For surgical applications, instrument readiness can be a matter of life and death. Yet, surgical instruments can be costly, making it inefficient to change out such instruments before replacement or maintenance is necessary.

### OVERVIEW

The present inventor recognizes the importance of tracking usage of surgical instruments to accurately determine tool life or a time between maintenance operations, such as sharpening or inspection. The present inventor further recognizes the importance of instrument use counters and methods that are accurate, inexpensive, and simple to use for greatest adoption by practitioners.

This patent document pertains generally to instrument use counters and methods for tracking instrument or tool use and more particularly to tracking the usage of surgical instruments. An instrument use counter can include a tray, an instrument holder, an actuator, and a counter. The tray can include an instrument holder positioned on or above an upper surface of the tray. The instrument holder can be configured to retain at least one instrument. The actuator can be located in proximity to the instrument holder and can include a proximity sensor in electronic communication with the at least one instrument or a moveable member engageable with the at least one instrument during removal from, or receipt by, the instrument holder. The counter can be coupled to the tray, positioned above the upper surface, and operably coupled to the proximity sensor or the movable member of the actuator. The counter can include a numerical readout member, whereby removal of the at least one instrument from, or receipt of the at least one instrument by, the instrument holder is indicated on the numerical readout member as a use.

To better illustrate the instrument use counters and methods disclosed herein, a non-limiting list of examples is provided here:

In Example 1, an instrument use counter can comprise an instrument holder, configured to retain at least one instrument, coupled to, or integrated with, a tray and positioned on or above an upper surface of the tray; an actuator, located in proximity to the instrument holder, including a proximity sensor in electronic communication with the at least one instrument or a movable member engageable with the at least one instrument during removal from, or receipt by, the instrument holder; and a counter, operably coupled to the proximity sensor or the movable member of the actuator, including a numerical readout member, whereby removal of the at least one instrument from, or receipt of the at least one instrument by, the instrument holder is indicated on the numerical readout member as a use.

In Example 2, the instrument use counter of Example 1 can optionally be configured to further comprise a reset member configured to change a value on the numerical readout member to zero.

In Example 3, the instrument use counter of any one or any combination of Examples 1 or 2 can optionally be configured such removal of the at least one instrument from, or receipt of the at least one instrument by, the instrument holder results in an electronic signal change of the proximity sensor or movement of the movable member of the actuator.

In Example 4, the instrument use counter of any one or any combination of Examples 1-3 can optionally be configured such that the movable member of the actuator is configured as a turnstile or a wheel.

In Example 5, the instrument use counter of any one or any combination of Examples 1-4 can optionally be configured such that the movable member of the actuator is configured as an arm.

In Example 6, the instrument use counter of any one or any combination of Examples 1-5 can optionally be configured such that the movable member of the actuator is configured as a button.

In Example 7, the instrument use counter of any one or any combination of Examples 1-6 can optionally be configured such that the tray, the actuator, and the counter include a reusable and sterilizable material.

In Example 8, the instrument use counter of any one or any combination of Examples 1-7 can optionally be configured such that the numerical readout member includes an electronic display.

In Example 9, the instrument use counter of any one or any combination of Examples 1-8 can optionally be configured such that a plurality of instrument holders are coupled to, or integrated with, the tray.

In Example 10, the instrument use counter of any one or any combination of Examples 1-9 can optionally be configured such that receipt of the at least one instrument by the instrument holder has no effect on the numerical readout member.

In Example 11, the instrument use counter of any one or any combination of Examples 1-10 can optionally be configured such that the counter includes a wireless transceiver configured to transmit data to a computer system.

In Example 12, the instrument use counter of any one or any combination of Examples 1-11 can optionally be configured such that the counter includes a wireless transceiver configured to receive data from a computer system.

In Example 13, a method of tracking usage of one or more surgical instruments, can comprise the steps of: placing the one or more surgical instruments into one or more instrument holders located on, or integrated with, a tray; removing a first surgical instrument from a first instrument holder, including engaging a proximity sensor or a movable member of a first actuator, located in proximity to the first instrument holder, and a portion of the first surgical instrument; reading a numerical readout member of a first counter, operably coupled to the proximity sensor or the movable member of the first actuator; and determining if the first surgical instrument has a remaining useful life based, at least in part, on a value displayed on the numerical readout member of the first counter.

In Example 14, the method of Example 13 can optionally be configured such that determining if the first surgical instrument has a remaining useful life includes comparing the value displayed on the numerical readout member with a value included in a predetermined lookup table of useful life values.

In Example 15, the method of any one or any combination of Examples 13 or 14 can optionally be configured to further comprise the step of sterilizing at least one of the tray, the first surgical instrument, the first instrument holder, or the first counter after a usage.

In Example 16, the method of any one or any combination of Examples 13-15 can optionally be configured to further comprise the step of wirelessly transmitting an instrument usage value from the first counter to a computer system.

In Example 17, the method of any one or any combination of Examples 13-16 can optionally be configured such that engaging the movable member of the first actuator and the portion of the first surgical instrument includes turning or translating the movable member.

In Example 18, the method of Example 17 can optionally be configured such that turning or translating the movable member includes increasing the value on the numerical readout member.

In Example 19, the method of any one or any combination of Examples 13-18 can optionally be configured to further comprise the steps of: removing a second surgical instrument from a second instrument holder, including engaging a proximity sensor or a movable member of a second actuator, located in proximity to the second instrument holder, and a portion of the second surgical instrument; reading a numerical readout member of a second counter, operably coupled to the proximity sensor or the movable member of the second actuator; and determining if the second surgical instrument has a remaining useful life based, at least in part, on a value displayed on the numerical readout member of the second counter.

In Example 20, the method of Example 19 can optionally be configured such that determining if the second surgical instrument has a remaining useful life includes comparing the value displayed on the numerical readout member with a value included in a predetermined lookup table of useful life values.

In Example 21, the instrument use counter or method of any one or any combination of Examples 1-20 can optionally be configured such that all elements, operations, or other options recited are available to use or select from.

These and other examples and features of the present instrument use counters and methods will be set forth in part in the following Detailed Description. This Overview is intended to provide non-limiting examples of the present subject matter—it is not intended to provide an exclusive or exhaustive explanation. The Detailed Description below is included to provide further information about the present instrument use counters and methods.

### BRIEF DESCRIPTION OF THE DRAWINGS

Corresponding reference characters indicate corresponding parts throughout the several views. The exemplifications set out herein illustrate exemplary embodiments of the invention, and such exemplifications are not to be construed as limiting the scope of the invention in any manner.

FIG. 1 illustrates a top view of an instrument use counter assembly, in accordance with at least one example of the present disclosure.

FIG. 2 illustrates a front view of the instrument use counter assembly of FIG. 1.

FIG. 3 illustrates a side view of the instrument use counter assembly of FIG. 1.

FIG. 4 illustrates a top view of an instrument use counter assembly, in accordance with at least one example of the present disclosure.

FIG. 5 illustrates a front view of the instrument use counter assembly of FIG. 4.

FIG. 6A illustrates a side view of the instrument use counter assembly of FIG. 4 and an instrument.

FIG. 6B illustrates a side view of the instrument use counter assembly of FIG. 4 with an instrument removed.

FIG. 7 illustrates a top view of an instrument use counter assembly, in accordance with at least one example of the present disclosure.

FIG. 8A illustrates a front view of the instrument use counter assembly of FIG. 7 and an instrument.

FIG. 8B illustrates a front view of the instrument use counter assembly of FIG. 7, with an instrument removed.

FIG. 9A illustrates a side view of the instrument use counter assembly of FIG. 7 and an instrument.

FIG. 9B illustrates a side view of the instrument use counter assembly of FIG. 7, with an instrument removed.

FIG. 10 illustrates a top view of an instrument tray containing multiple instrument holders, instruments, and instrument counters, in accordance with at least one example of the present disclosure.

FIG. 11 illustrates a top view of an instrument tray containing multiple instrument insert holders, instruments, and instrument counters, in accordance with at least one example of the present disclosure.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

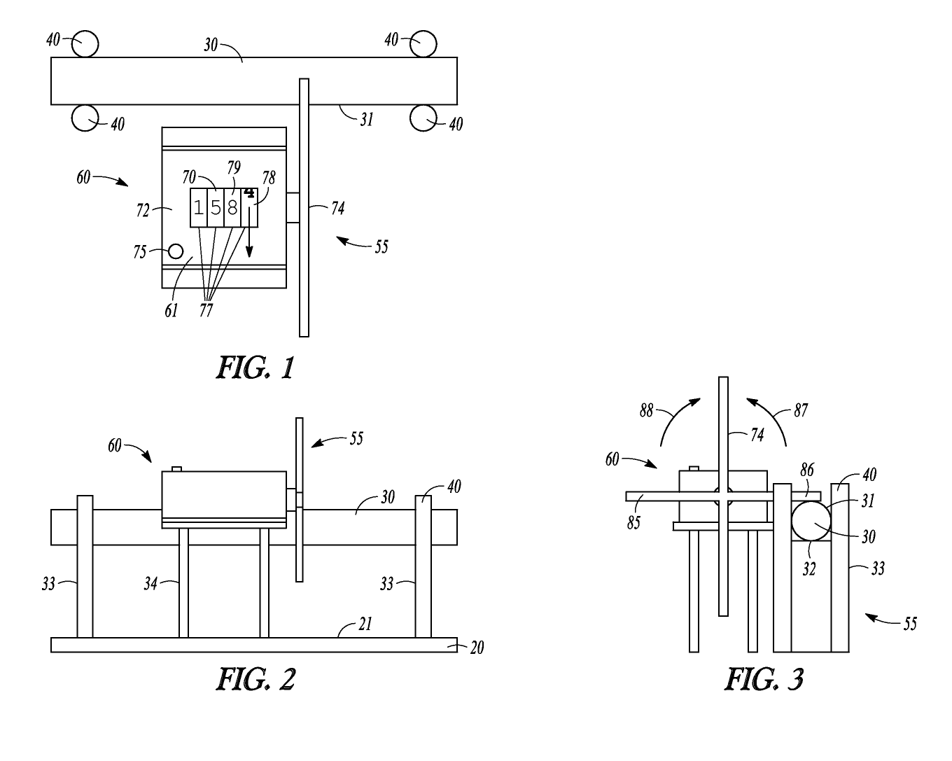

FIG. 1 illustrates a top view of an instrument use counter assembly 55, which can include a counter 60 and an instrument holder 40 configured to retain an instrument 30.

The counter 60 can include a counter housing 72 that can act as a covering and provide protection for moving parts within the counter 60. In an example, the counter housing 72 can include a material impermeable to high temperature sterilization fluids used to sterilize surgical equipment. The counter housing 72 can be sealed to protect against liquid or heat damage from a sterilization process. The counter housing 72 can be shaped as a box (as shown) or cylindrical, spherical, or any other regular or irregular polygonal shape. The counter 60 can have a top face 61 including a numerical readout member 70. The numerical readout member 70 can be configured to correspond to an estimated number of uses in a monitored period. In an example, the counter 60 can be designed for a maximum of 99 uses and can have two digits in the numerical readout member 70. The numerical readout member 70 can be configured for any number of maximum uses and can have as many digits as are required. The numerical readout member 70 can be located on any face or portion of the counter 60. The numerical readout member 70 can be located so that it can be visible and read easily by a surgeon, nurse, technician or other practitioner.

The instrument use counter assembly 55 can further include an actuator 74. The actuator can be connected to a series of disks 77 or rings containing numerals. The connection can be configured as a shaft including gears and/or a ratcheting mechanism. The disks can be configured to correspond to a column of numerals, such as a ones disk 78, a tens disk 79 and so forth. When the ones disk 78 reaches the number 9, and the instrument 30 is removed from the instrument holder 40, a latching mechanism can turn the tens disk 79 one notch and the ones disk 78 will be reset to zero. In another example, the counter 60 can be configured as an electronic display.

The ratcheting mechanism can be configured so that when an instrument is removed from an instrument holder 40, the numerical readout member 70 will register one removal. The counter 60 can be configured so that when the instrument is returned to the instrument holder 40, the counter 60 will ratchet and the numerical readout member 70 will not change.

Alternatively, the counter 60 can be configured in the reverse of the previous manner. In this configuration, replacement of the instrument 30 into the instrument holder 40 will cause the counter 60 to record one use on the numerical readout member 70 and removal of the instrument 30 from the instrument holder 40 will ratchet the actuator 74 and the value on the numerical readout member 70 will not change.

The actuator 74 can be configured as a single longitudinal member, a turnstile, a wheel, a button, or a lever, for example, to actuate the counter 60. The counter 60 can include a reset member 75. The reset member 75 can be configured as a button, a switch, a lever or an actuator and, when actuated, can reset the numerical readout member 70 to a zero value. The counter 60 can be reset to zero when a refurbished, new, or inspected instrument is placed in the instrument holder 40. The actuator 74 can be configured to be moveable when engaged by the instrument 30. The engagement can take place upon either or both of removal of the instrument 30 from the instrument holder 40 or replacement of the instrument 30 to the instrument holder 40. The movement of the actuator 74 can be a rotational movement, a sliding movement, a bumping movement, a pressure, or it could be a movement detected by an electronic field such as in a Hall Effect sensor or an electronic proximity sensor. The engagement of the instrument 30 and the actuator 74 can take place on any surface of the instrument. In an example, the actuator 74 can be positioned in proximity to an instrument's upper surface 31.

FIG. 2 illustrates a front view of the instrument use counter assembly 55 of FIG. 1. The instrument holder 40 can be configured as a series of support members 33. The support members 33 can be post-like in shape and can be attached to an upper surface 21 of an instrument tray 20. The support members 33 can provide a snap-fit for instruments 30. The support members can provide a framework for holding the instrument 30 securely and can be individualized to fit only a particular instrument, such that if a surgical tray were configured to hold more than one instrument, the instruments would not be interchangeable in the instrument holders 40. In the alternative, instruments 30 and instrument holders 40 can be numbered, coded, color coded, or marked so that each individual instrument 30 is always returned to the instrument holder 40 assigned to that particular instrument. In an example, the instrument holder 40 can include a silhouette of a corresponding instrument 30.

The counter 60 can be connected to counter supports 34, which can act to attach the counter 60 to the tray 20 and provide a proper height for the counter 60 relative to the instrument 30 when located in the instrument holder 40. The relative sizes of the counter 60 to the instrument 30 in FIGS. 1-11 are for illustration only and the counter 60 can be miniaturized and located in relation to the instrument holder 40 in a configuration designed to maximize space in the tray 20.

FIG. 3 illustrates a side view of the instrument use counter assembly 55 of FIG. 1. The actuator 74 can be configured as a turnstile 85. The turnstile 85 can include more than one arm 86. An arm 86 can be positioned in close proximity to an instrument 30 so that the arm 86 will be engaged when the instrument 30 is removed from an instrument holder 40. The engagement of the arm 86 by the instrument 30 can cause the actuator 74 to move. The movement of the actuator 74 in the removal direction 87 can cause the counter 60 to register one removal or use of the instrument 30.

When a surgeon has finished using an instrument 30, replacement of the instrument 30 into the instrument holder 40 can cause the actuator 74 to move. Movement of the actuator in the replacement direction 88 may not cause any registration on the numerical readout member 70 (see FIG. 1). In an example, the actuator 74 can ratchet in the replacement direction 88. The actuator 74 can include spring loading and positional detents that cause the actuator to return to a set position when the instrument 30 is returned to an instrument holder 40. In an example, the arm 86 can be in close proximity to the instrument's upper surface 31. In another example, the arm 86 can be in close proximity to the instrument's lower surface 32, such that removal of the instrument 30 from the instrument holder 40 can cause the arm 86 to move.

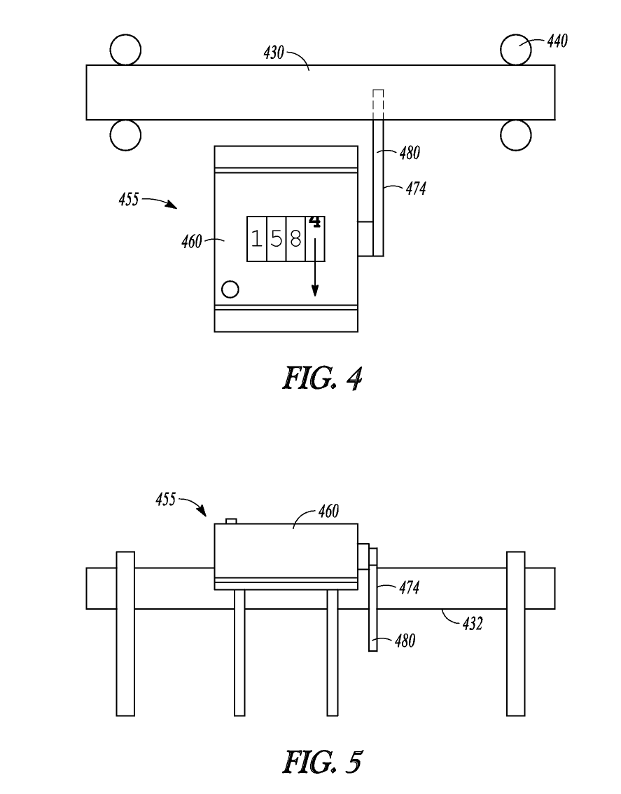

FIG. 4 illustrates an example of an instrument use counter assembly 455. A counter 460 can be operatively coupled with an actuator 474 including a lever arm 480. The lever arm 480 can be positioned and configured to engage an instrument 430 when it has been placed in an instrument holder 440.

FIG. 5 illustrates a front view of the instrument use counter assembly 455 of FIG. 4. The lever arm 480 of the actuator 474 can engage the instrument's lower surface 432.

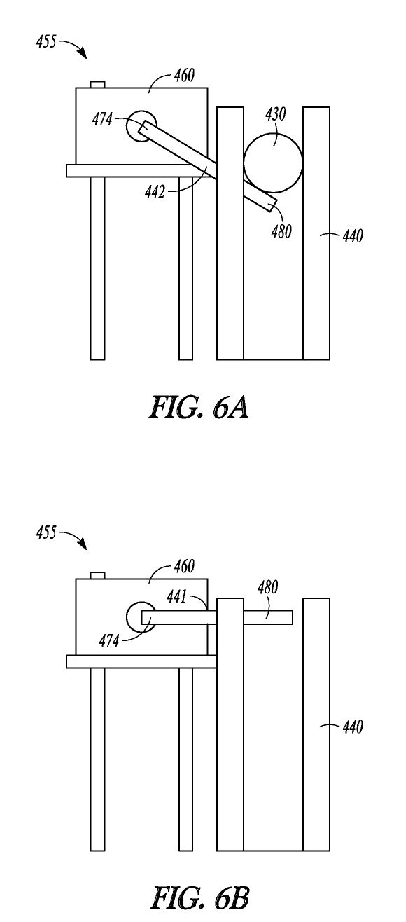

FIG. 6A is an illustration of a side view of the instrument use counter assembly 455 of FIG. 4. The lever arm 480 of the actuator 474 can move downwardly when an instrument 430 is placed in the instrument holder 440 so that the lever arm 480 assumes a loaded position 442. The actuator 474 can be configured to record a use value upon the downward movement of the lever arm 480 when an instrument 430 is loaded into the instrument holder 440. Alternatively, the actuator 474 can be configured to record a use value upon an upward movement of the lever arm 480 caused by a resilient member, such as a spring, in engagement with the actuator 474. The actuator 474 can be spring loaded so that in FIG. 6B, the lever arm 480 assumes an unloaded position 441 when the instrument holder 440 is empty. The lever arm 480 can produce movement of the actuator 474 when an instrument 430 is placed in the instrument holder 440, moving from the unloaded position 441 to the loaded position 442 (see FIG. 6A). The lever arm 480 can produce movement of the actuator 474 when an instrument 430 is removed from the instrument holder 440 moving from the loaded position 442 (see FIG. 6A) to the unloaded position 441.

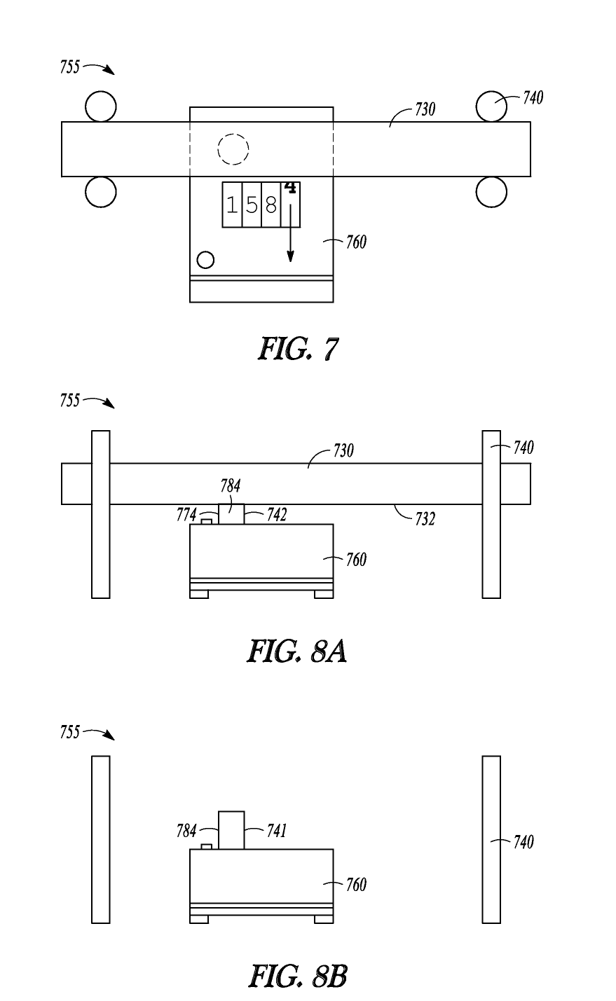

FIG. 7 illustrates an example of a top view of an instrument use counter assembly 755. This example shows that placement of an actuator 774 or a counter 760 can vary. Placement of the counter 70 or actuator 774 can be substantially underneath an instrument 730 as it is located in a respective instrument holder 740.

FIG. 8A is a front view of the instrument use counter assembly 755 of FIG. 7. As the instrument 730 occupies the instrument holder 740, the instrument's lower surface 732 can engage the actuator 774. The actuator 774 can take the form of a switch, lever, arm, turnstile, wheel or button, for example. In an example, a button 784 can assume a loaded position 742 when the instrument 730 occupies the instrument holder 740.

FIG. 8B is an illustration of the actuator 774 and the instrument holder 740 when the instrument 730 has been removed from the holder. The button 784 can be biased by a resilient member, such as a spring, and will assume a raised unloaded position 741 when no longer depressed by the instrument 730.

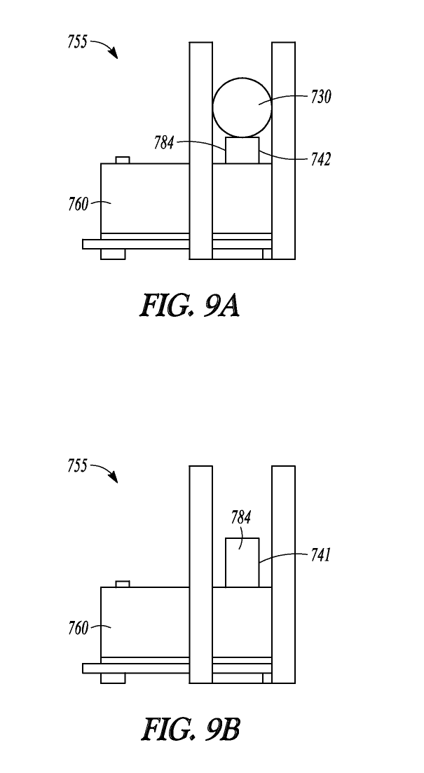

FIGS. 9A and 9B show the button 784 in the loaded position 742 and the unloaded position 741 from a side view of the instrument use counter assembly 755 of FIG. 7. The counter 760 or actuator 774 can be placed substantially underneath the instrument 730.

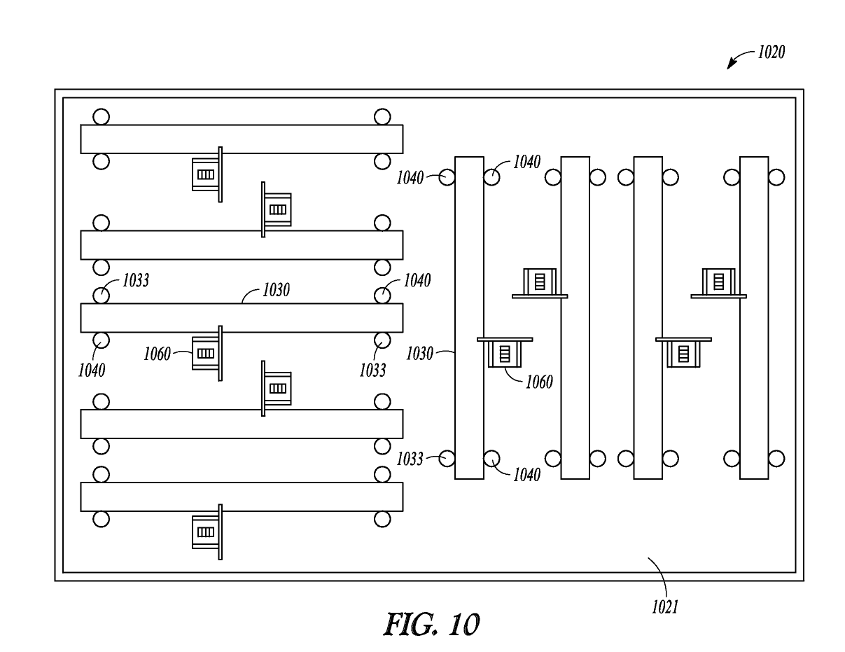

FIG. 10 illustrates a top view of an instrument tray 1020 containing multiple instrument holders 1040, instruments 1030, and counters 1060. Each individual instrument holder 1040 can be marked, coded, or designed to accept a specific instrument 1030 in order to accurately tally the uses of each individual instrument 1030. The tray 1020 can include instrument holders 1040 similar to those described in FIGS. 1-9.

The instrument tray 1020 can include an upper surface 1021 coupled to one or more instrument holders 1040 in the form of support members 1033. The support members 1033 can be configured as posts attached to the upper surface 1021 and having holding members providing a snap fit for instruments 1030. The support members 1033 can provide a framework for securely holding an instrument and can be individualized to fit only a particular instrument 1030, such that if a surgical tray were configured to hold more than one instrument, the instruments would not be interchangeable in the support members 1033. Additionally or alternatively, the instrument holders 1040 can be configured in any configuration known to those skilled in the art. Additionally or alternatively, instruments 1030 and instrument holders 1040 can be numbered, coded, or marked so that each individual instrument is always returned to an instrument holder assigned to that particular instrument.

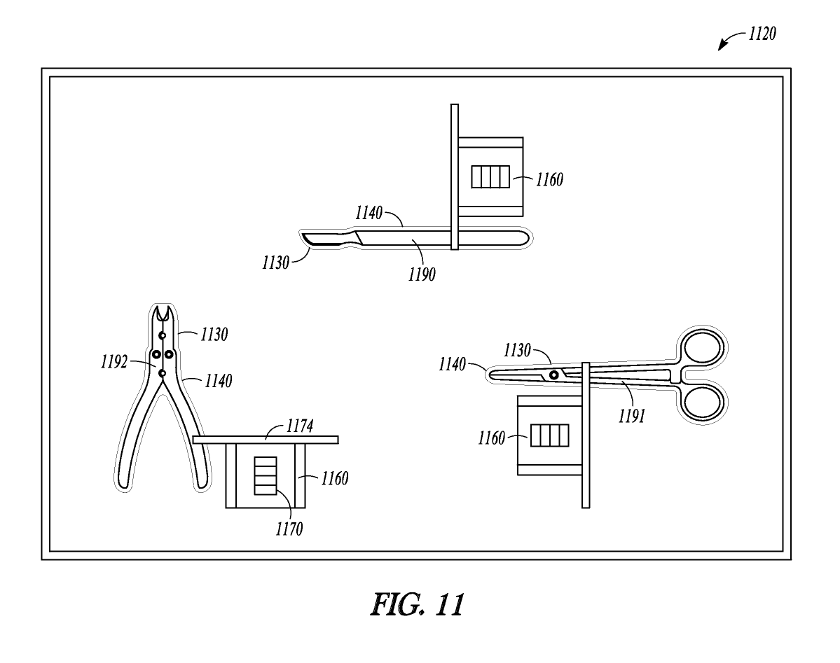

FIG. 11 illustrates a top view of an instrument tray 1120 including multiple instrument holders 1140, instruments 1130, and instrument counters 1160. The instrument holders 1140 can be in the form of a molded cut-out that is form fit to a portion of a shape of the instruments 1130, for example a scalpel 1190, a forceps 1191, and a cutting tool 1192. As in the previous examples, an actuator 1174 can be located and configured such that removal of the instrument 1130 will engage a portion of the actuator 1174 and a numerical readout member 1170 will register a use of the instrument 1130.

Any of the instrument use counter examples disclosed in this patent document can be configured with a wireless transceiver or an electronic counting module, such that movement or electronic proximity sensing of an actuator can be transformed into digital data. The transceiver can send information to, and receive information from, a computer system configured to track instrument usage. The computer system can be connected to a network such as the internet and instrument use can be monitored, tracked, or logged remotely.

The above Detailed Description includes references to the accompanying drawings, which form a part of the Detailed Description. The drawings show, by way of illustration, specific embodiments in which the present instrument use counters and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, a device, assembly, kit, or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. An instrument use counter, comprising:
an instrument holder, configured to retain at least one instrument, coupled to, or integrated with, a tray and positioned on or above an upper surface of the tray;
an actuator, located in proximity to the instrument holder, including a proximity sensor in electronic communication with the at least one instrument or a movable member engageable with the at least one instrument during removal from, or receipt by, the instrument holder; and
a counter, operably coupled to the proximity sensor or the movable member of the actuator, including a numerical readout member,
whereby removal of the at least one instrument from, or receipt of the at least one instrument by, the instrument holder is indicated on the numerical readout member as a use.

2. The instrument use counter of claim 1, further comprising a reset member configured to change a value on the numerical readout member to zero.

3. The instrument use counter of claim 1, wherein removal of the at least one instrument from, or receipt of the at least one instrument by, the instrument holder results in an electronic signal change of the proximity sensor or movement of the movable member of the actuator.

4. The instrument use counter of claim 1, wherein the movable member of the actuator is configured as a turnstile or a wheel.

5. The instrument use counter of claim 1, wherein the movable member of the actuator is configured as an arm.

6. The instrument use counter of claim 1, wherein the movable member of the actuator is configured as a button.

7. The instrument use counter of claim 1, wherein the tray, the actuator, and the counter include a reusable and sterilizable material.

8. The instrument use counter of claim 1, wherein the numerical readout member includes an electronic display.

9. The instrument use counter of claim 1, wherein a plurality of instrument holders are coupled to, or integrated with, the tray.

10. The instrument use counter of claim 1, wherein receipt of the at least one instrument by the instrument holder has no effect on the numerical readout member.

11. The instrument use counter of claim 1, wherein the counter includes a wireless transceiver configured to transmit data to a computer system.

12. The instrument use counter of claim 1, wherein the counter includes a wireless transceiver configured to receive data from a computer system.

13. A method of tracking usage of one or more surgical instruments, comprising:
placing the one or more surgical instruments into one or more instrument holders located on, or integrated with, a tray;
removing a first surgical instrument from a first instrument holder, including engaging a proximity sensor or a movable member of a first actuator, located in proximity to the first instrument holder, and a portion of the first surgical instrument;
reading a numerical readout member of a first counter, operably coupled to the proximity sensor or the movable member of the first actuator; and
determining if the first surgical instrument has a remaining useful life based, at least in part, on a value displayed on the numerical readout member of the first counter.

14. The method of claim 13, wherein determining if the first surgical instrument has a remaining useful life includes comparing the value displayed on the numerical readout member with a value included in a predetermined lookup table of useful life values.

15. The method of claim 13, further comprising sterilizing at least one of the tray, the first surgical instrument, the first instrument holder, or the first counter after a usage.

16. The method of claim 13, further comprising wirelessly transmitting an instrument usage value from the first counter to a computer system.

17. The method of claim 13, wherein engaging the movable member of the first actuator and the portion of the first surgical instrument includes turning or translating the movable member.

18. The method of claim 17, wherein turning or translating the movable member includes increasing the value on the numerical readout member.

19. The method of claim 13, further comprising:
removing a second surgical instrument from a second instrument holder, including engaging a proximity sensor or a movable member of a second actuator, located in proximity to the second instrument holder, and a portion of the second surgical instrument;
reading a numerical readout member of a second counter, operably coupled to the proximity sensor or the movable member of the second actuator; and
determining if the second surgical instrument has a remaining useful life based, at least in part, on a value displayed on the numerical readout member of the second counter.

20. The method of claim 19, wherein determining if the second surgical instrument has a remaining useful life includes comparing the value displayed on the numerical readout member with a value included in a predetermined lookup table of useful life values.
